(19) United States
(12) Patent Application Publication
(10) Pub. No.: US 2017/0219505 A1
(43) Pub. Date: Aug. 3, 2017

(54) METHOD FOR TESTING A CERAMIC COMPONENT

(71) Applicant: Aktiebolaget SKF, Goteborg (SE)

(72) Inventor: Oskar Schoeppl, Linz (AT)

(21) Appl. No.: 15/415,306

(22) Filed: Jan. 25, 2017

(30) Foreign Application Priority Data
Feb. 3, 2016 (DE) 102016201647.4

(51) Int. Cl. G01N 25/72; G01N 33/38; C04B 35/48; C04B 35/597; C04B 35/565; C04B 35/10; G01N 21/88; C04B 35/584

(52) U.S. Cl. CPC G01N 25/72; G01N 21/8803; G01N 33/388; C04B 35/584; C04B 35/597; C04B 35/565; C04B 35/10; C04B 35/48; C04B 2235/3873; C04B 2235/3869; C04B 2235/3826; C04B 2235/3217; C04B 2235/3244; C04B 2235/9607

(57) ABSTRACT

A method for testing a ceramic component for a fracture toughness includes changing the temperature of the component to a first temperature, for example, by heating the component, and changing the temperature of the component to a second temperature, for example, by cooling the component and testing the component for cracks. The temperature difference between the first temperature and the second temperature is determined based on a minimum fracture toughness.

### CROSS-REFERENCE

This application claims priority to German patent application no. 10 2016 201 647.4 filed on Feb. 3, 2016, the contents of which are fully incorporated herein by reference.

### TECHNOLOGICAL FIELD

Exemplary embodiments relate to a method for testing a ceramic component for a suitability for use as well as to a component tested using this method.

### BACKGROUND

Ceramic components are used in a variety of applications, for example, in rolling-element bearings, as sliding bearings, as rolling elements, as cutting elements, as implants, or the like. High mechanical loads or temperatures often act on the components, for example, on the surface.

Under certain circumstances the component can have cracks or defects on the surface, but also in other regions. These defects can be present for different reasons, for example, having arisen in a manufacturing or in a post-processing, such as grinding, honing, or the like. The imperfections can cause the ceramic component to fail under stress. Under a load the crack can grow and lead to an unwanted material failure.

In order to prevent this there are conventional test methods, using which methods defective components are to be eliminated. For example, as proposed in DE 10 2010 017 351 A1, for this purpose the components are subjected to a thermal shock treatment. For this purpose the ceramic components are subjected to a rapid heating within a short time in order to bring defective components to failure in a targeted manner A detection of arising cracks is effected in an acoustic manner during a cooling. If a growth of a crack is detected, the component is eliminated. However, under unfavorable conditions the bench test can be relatively unspecific. It can possibly happen that components are also eliminated that are usable because no distinction is made between critical and non-critical cracks.

### SUMMARY

There is therefore a need to improve a test method for a ceramic component. This need is taken into account by the method and the component according to the disclosure.

Exemplary embodiments relate to a method for testing a ceramic component for a suitability for use. For this purpose the component is heated or cooled to a first temperature. The component is subsequently heated or cooled to a second temperature. A temperature difference between the first temperature and the second temperature is determined based on a minimum fracture toughness. The component is subsequently tested for cracks.

The minimum fracture toughness can be, for example, a value prescribed by an operator or user who is to have the component in order to fulfill the intended use.

The temperature change can be, for example, a heating or a cooling of the component. Under certain circumstances the second temperature can be lower than the first temperature. The temperature change can be effected, for example, in a gas, for example, in air, for example, in an oven and/or in a liquid, for example, in a bath, but also with radiation, for example, using a laser. The first temperature change can possibly be a heating that is effected in an oven, and the second temperature change can be a cooling that is effected in a liquid bath, for example, in water or in oil. However, a temperature change, for example, a heating, but also a cooling, can be effected here under any configurable atmosphere, but also under vacuum.

A testing of the component for cracks can be effected in any manner that is suited to detect any cracks at all or critical cracks, for example, optically, acoustically, and/or haptically. “Optically” can mean, for example, that the crack is detectable with the naked eye or with a camera. In some exemplary embodiments a threshold value can then be established for a size starting from which a crack is considered critical. The threshold value can be, for example, a length, a width, and/or a depth of the crack. A threshold value for the size can be, for example, at least 0.01 mm, a threshold value for the depth, for example, at least 0.01 mm, and/or a threshold value for the width, for example, at least 0.01 mm. Additionally or alternatively detection methods can also be used with which only cracks can be detected that exceed the corresponding threshold value. A crack can be, for example, a defect that deviates from a desired condition of the component. This defect can arise in the thermal shock treatment or, however, also have already have been present prior to the thermal shock treatment. The defect or the crack can possibly only have exceeded a critical size due to the thermal shock treatment. A “thermal shock treatment” can mean, for example, a impulsive, i.e., occurring in a short time, temperature increase or cooling of a ceramic component. A short time duration can last, for example, shorter than 1 hour, 50 minutes, or 30 minutes, and/or at least 10 seconds, 20 seconds, or 30 seconds.

The ceramic component can be all possible components that at least partially comprise or are manufactured from a ceramic material, for example, an implant for a human or animal body, for example, a joint, a bone screw, a dental prosthesis, a dental bridge, or the like. Furthermore the component can be a bearing component for a rolling-element- or sliding-bearing, for example, a bearing ring, or a rolling element, or in general for rolling-element, rolling, and sliding applications. In addition, the components can also be a valve, a nozzle body, a cutting element, a ceramic circuit board, a functional component, or the like. Some of the components here can also contain metallic or organic components. With all of these components a failure can be unwanted and lead to significant rework and possibly operations. In some exemplary embodiments at least the risk that a failure occurs can be reduced by the method or the use of components that have been tested using the method.

A minimum fracture toughness can be determined here, for example, based on experience values, experiments, or the like. A minimum required fracture toughness can be, for example, 4.0 MPa m^{1/2}.

The temperature difference can be determined, for example, starting from the Griffith/Irwin criterion:

K≧K_{Ic} with K=σ_{Ref} Y √aπ

K here stands for the stress intensity factor. K_{Ic} represents the critical stress intensity factor. σ_{Ref} stands for a reference stress in a test without crack, “a” stands for the size of the crack, and Y represents a geometric factor, using which the geometry of the crack, of the stress field, and of the test body is taken into account.

The thermally induced stress σₜₕ or its maximum value σ_{th,max} on a surface under tensile stress can generally be represented by the following equation:

$${σ}_{th , max} = {\hat{σ}}_{th}^{*} \frac{α E}{1 - v} Δ T$$

α is the linear thermal expansion coefficient, E stands for the E modulus for the Poisson ratio, and ΔT for the temperature difference in the thermal shock treatment. The dimensionless factor \hat{σ}*ₜₕ refers to the component geometry and a quenching parameter. This factor falls between the value 0 with very gentle cooling and the value 1 for a very rapid quenching process.

For the specific test with the presence of a crack with the length a the stress intensity factor K can be represented as follows:

$$K = Δ {T}_{c} \frac{α eff E}{1 - v} {σ}_{th , max}^{*} {Y}_{max} √ a π$$

Yₘₐₓ here is the geometry factor Y in a certain point of the crack. This is dependent on the crack shape and can be determined by a parameter study. Extending of a crack or of a defect occurs when corresponding to equation the fracture toughness K of the material is reached at the location of the crack or defect. By reformulating the above equation, for idealized conditions such as temperature-dependent material parameters and low stress changes over the course of the crack it results that with prescribed test geometry and required fracture toughness a certain critical crack size corresponds to a certain critical temperature difference ΔT_{c} in the thermal shock treatment. Under non-idealized conditions this temperature difference can be numerically calculated. If the temperature difference is set accordingly, in some exemplary embodiments it can be ensured that all components that withstand the thermal shock treatment with the specified temperature difference are suited for use in operation or have the minimum required fracture toughness. Components wherein cracks arise in the testing that exceed a threshold value can be eliminated.

Additionally or alternatively, taking into consideration the component-specific and systematic errors for determining the fracture toughness of rolling-element bearing balls made from silicon nitride, the estimated measurement error for the calculated fracture toughness can at most fall in the range of +/−10%. In some exemplary embodiment the components tested using the method can have a sufficient safeness against failure.

Additionally or alternatively a value for the fracture toughness can be taken from a table and/or experimentally determined.

In order to determine a fracture toughness experimentally, a fracture toughness can be determined, for example, on a so-called test component using the following method. For example, the test component can have the same shape, dimensions, and material properties as the ceramic component.

For this purpose a semi-elliptical surface crack is introduced using, for example, a

Knoop indenter for the purpose of good reproducibility of the crack geometry. In the table below a Knoop indent having the load 10 kg and 7 kg has been introduced (HK10, HK7) on silicon nitride rolling-element bearing balls having different diameter D. The plastically deformed material at the component surface is preferably removed by ablating of a thin surface layer in order to reduce the influence on the measurement result of introduced residual stresses. The ablated layer should be at least ⅙ of the longer diagonal of the Knoop indenter. By introducing a specific temperature difference T in the thermal shock experiment a fracture toughness Ksurv is obtained, just at the point where still no crack growth is yet detectable. With further increase of T crack growth results with the fracture toughness Kfrac. Here the critical fracture toughness Kc that correlates to ΔTc according to equation has already been exceeded. According to the invention the heat transfer coefficient hf with quenching of ceramics in water falls in the range of 75000 to 100000 Wm⁻²K⁻¹. Bi is a dimensionless Biot number from the modeling of the temperature field of the ball. The fracture toughness calculated in this manner can also be referred to, for example, as the measured fracture toughness.

| | h_{f} = 75000 Wm⁻²K⁻¹ | | | h_{f} = 100000 Wm⁻²K⁻¹ | | |
|---|---|---|---|---|---|---|
| | Bi | Kₛᵤᵣᵥ | K_{frac} | Bi | Kₛᵤᵣᵥ | K_{frac} |
| | — | MPa m^{1/2} | MPa m^{1/2} | — | MPa m^{1/2} | MPa m^{1/2} |
| Set 1, D = 12.7 mm, HK10 | 22.0 | 5.3 ± 0.3 | 5.5 ± 0.3 | 29.3 | 5.7 ± 0.3 | 5.9 ± 0.3 |
| Set 2, D = 5.55 mm, HK10 | 10.2 | 5.5 ± 0.3 | 5.9 ± 0.3 | 13.7 | 6.0 ± 0.3 | 6.4 ± 0.3 |
| Set 3, D = 5.55 mm, HK7 | 10.6 | 5.7 ± 0.1 | 6.0 ± 0.2 | 14.2 | 6.2 ± 0.1 | 6.5 ± 0.2 |

In some exemplary embodiments the calculating of the fracture toughness is integrated into the method for testing. In some exemplary embodiments more precise results can possibly be determined thereby. For example, the determining of the fracture toughness can be effected in the same device for changing the temperature of the object, at the same location, and/or in a time interval that has at most 1/10 of a total length of the method for testing. For example a proportion of a total amount of ceramic components to be tested can be provided as so-called test components with a crack for determining the fracture toughness according to the described method. These test components can then be eliminated later and not used for an application. The total quantity of ceramic components to be tested can be, for example, a quantity of more than 1000 ceramic components. A portion of test components that are provided with a crack for determining the fracture toughness can be removed from the total quantity. The portion can be, for example, at least 10, 15, 20, or thirty ceramic components. The portion can be, for example, smaller than 100, 90, 80, or 70 ceramic components.

Additionally or alternatively, the optical checking of the component for cracks can be effected using a black dye. Here it can be for example a crack-penetrating dye and/or a printer dye. In some exemplary embodiments cracks in components that comprise Al₂O₃ or ZrO₂ can be sufficiently well recognized. Depending on the material of the component the dye used can possibly be different; with Si₃N₄ a fluorescent dye can be used, for example, that fluoresces in UV light.

Additionally or alternatively the method can also be performed with components that comprise a ceramic material other than Si₃N₄, for example, SiAlON (silicon aluminum oxynitride), SiC (silicon carbide), Al₂O₃ (aluminum oxide), ZrO₂ (zirconium oxide), ZTA (zirconium-oxide reinforced aluminum oxide), ATZ (aluminum-oxide reinforced zirconium oxide), ATZ (aluminum-oxide reinforced zirconium oxide), or the like.

Exemplary embodiments also relate to a ceramic component that has been tested using a method according to one of the preceding claims for a suitability for use. In some exemplary embodiments it can thereby be made possible that the components have maximum cracks that are smaller than the threshold value. In some exemplary embodiments a component can thus be provided wherein a failure in an intended operation is at least unlikely.

In addition, the ceramic component can comprise as material Si₃N₄, SiAlON, SiC, Al₂O₃, ZrO₂, or their mixtures. Under certain circumstances at least one of these materials can constitute at least 50% by weight of a total weight of the component. The component can possibly also be completely manufactured from one of these materials.

Additionally or alternatively the ceramic component can have a fracture toughness that is greater than or equal to 4 MPa√m. In some exemplary embodiments particularly resistant components can thereby be provided.

Additionally or alternatively the ceramic component can have a roughness at least sectionally on its surface that is less than 15 μm, 10 μm, 5 μm, 1 μm, 0.8 μm, 0.5 μm, 0.2 μm, 0.1 μm, 0.05 μm, 0.05 μm, 0.01 μm, 0.008 μm, 0.005 μm, or 0.001 μm. In some exemplary embodiments the components can thereby be particularly formed for their application purpose, for example as a bearing component or as an implant. Additionally or alternatively the component can have the roughness on its entire surface or on a surface that constitutes at least 50%, 60%, 70%, 80%, 90%, 95%, or 99% of a total surface of the component.

The exemplary embodiments and their individual features disclosed in the above description, the following claims, and the accompanying Figures can be meaningful and implemented both individually and in any combination for the realization of an exemplary embodiment in its various designs.

### BRIEF DESCRIPTION OF THE DRAWINGS

The Figures thus schematically show the following views.

FIG. 1 shows a schematic depiction of a method for testing a ceramic component according to an exemplary embodiment;

FIG. 2a shows a schematic depiction of a component according to an exemplary embodiment; and

FIG. 2b shows a schematic depiction of a component according to a further exemplary embodiment.

### DETAILED DESCRIPTION

In the following description of the accompanying Figures, like reference numbers refer to like or comparable components. Furthermore, summarizing reference numbers are used for components and objects that appear multiple times in an exemplary embodiment or in an illustration, but that are described together in terms of one or more common features. Components or objects that are described with the same or summarizing reference numbers can be embodied identically, but also optionally differently, in terms of individual, multiple, or all features, their dimensions, for example, as long as the description does not explicitly or implicitly indicate otherwise.

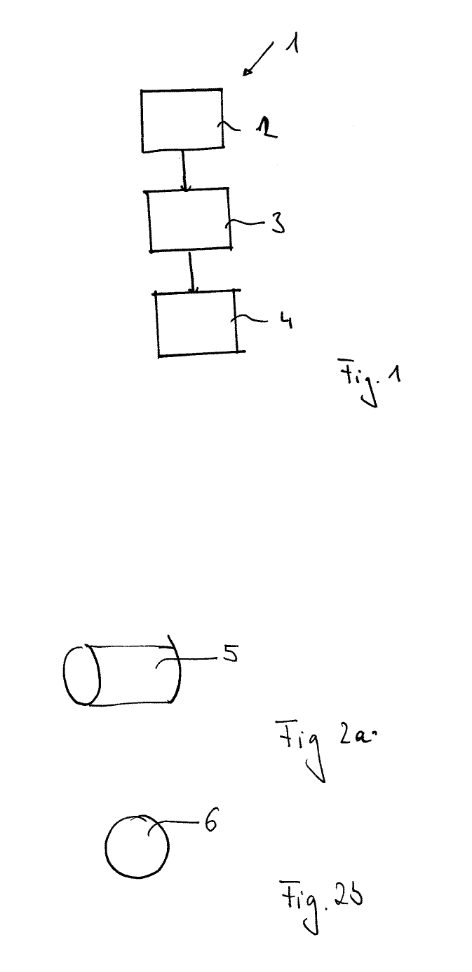

FIG. 1 shows a method 1 for testing a ceramic component for a fracture toughness. For this purpose in a first step 2 the component is heated or cooled to a first temperature. In a second step 3 the component is subsequently heated or cooled to a second temperature. A temperature difference between the first temperature and the second temperature is determined here based on a minimum fracture toughness. In a third step 4 the component is tested for fracture toughness.

In some exemplary embodiments the component can be heated at the first temperature until the heated regions or the entire component homogeneously has the first temperature. The component can subsequently be rapidly cooled. In some exemplary embodiments simple geometric framework conditions for calculating the temperature difference can thereby be obtained. Alternatively the setting of a constant or homogeneous first temperature in the component to be tested can be omitted. For example, only the surface or an edge layer can be heated to the first temperature and subsequently quickly cooled.

In exemplary embodiments wherein the thermal shock treatment or the test method is comprised of a rapid heating, i.e., the first temperature is lower than the second temperature, a homogeneous heating and cooling of the components or only a heating and cooling of the edge layers can be carried out. A rapid heating can be selected, for example, with components wherein in an operation internal tensile stresses can occur that can lead to a compressive stresses in an outer region. With components wherein only one point especially loaded in use is to be tested, only this point of the component may possibly be treated with the first temperature and then the second temperature. For example, a zonal rapid heating can be effected using a powerful laser.

Different approaches can be chosen to detect the cracks in the third step 4. In principle the cracks can be detected in any manner, for example, acoustically, by vibration analysis, or optically. Cracks that do not extend to the surface can be detected, for example, by ultrasound or X-rays.

For an optical testing the cracks can possibly be made recognizable using a crack-penetrating dye. Here the component to be tested is immersed in a dyed, for example, black, liquid, for example, printer dye. The dyed liquid remains in the cracks after removal of the body from the liquid. It can possibly also be a fluorescent crack-penetrating dye. The cracks can then be made visible, for example, under ultraviolet light. No matter by which method the cracks are detected, components that have cracks that exceed at least one threshold value can be eliminated.

The method according to exemplary embodiments can be suited, for example, to be used in a running production. For this purpose it can be provided that the components are heated during the production processes to the first temperature. For this purpose, for example, a conveyor can be provided, using which the component is transported through the oven. The ceramic component can subsequently be guided using the conveyor to a quenchant in order to be cooled to the second temperature. A liquid, for example, water or oil, is suitable as quenchant, for example. Furthermore a gas can also be used as quenchant. With a quenching with gas the gas can be guided to the body under increased pressure, for example under a pressure of 2 bar or higher. In other exemplary embodiments, in the first step 2 the component can also be zonally or entirely heated using a gas burner and subsequently cooled using an air jet in the second step 3.

If the ceramic component is guided using a conveyor to the liquid as quenchant, the components can be allowed to fall into the liquid in order to achieve the thermal shock treatment. Here the liquid has the second temperature. Alternatively the component can also be plunged into the liquid.

In some exemplary embodiments the temperature change to the first and the second temperature can be carried out in a single device, for example, when a gas is worked with for a quenching from a first temperature. For this purpose a combined heating- and quenching-system can be used. One example therefor is the heating in a vacuum hardening unit, wherein the heating can be performed with or without vacuum, possibly a heating with subsequent high-pressure-gas quenching.

In some exemplary embodiments an inhomogeneous temperature field on or in the component can be set during the temperature change process. In some exemplary embodiments a rapid heating of the component in combination with a rapid cooling can thereby be achieved. The thermal shock treatment can pertain to a near-surface zone of the component or the entire component. It is also possible here that the thermal shock treatment is carried out in a region wherein a maximum stress of the component occurs in use. Under certain circumstances edges of a body can also be subjected to the thermal shock treatment.

FIGS. 2a and 2b show schematic depictions of components 5 and 6 that are tested using the method 1. The component 5 is a cylindrical roller, and the component 6 a ball. The components 5 and 6 can, for example, serve as rolling elements. The components 5 and 6 include as ceramic material at least one of the materials Si₃N₄, SiAlON, SiC, Al₂O₃, ZrO₂, ZTA, or ATZ.

However, all possible other, for example, ceramic, components can be tested using the method 1. These can be, for example, components that are installed in rolling-element-, roller-, or sliding-applications. For this purpose the components are subjected as described to a test using thermal shock and subsequently impinged, for example, with crack-penetrating dye, in order to detect supercritically long cracks and thus to be able to eliminate components that are damaged or not suitable for use.

The exemplary embodiments and their individual features disclosed in the above description, the following claims, and the accompanying Figures can be meaningful and implemented both individually and in any combination for the realization of an exemplary embodiment in its various designs. In some further exemplary embodiments, features that are disclosed in other exemplary embodiments as device features can also be implemented as method features. Furthermore, features that are implemented in some exemplary embodiments as method features can also optionally be implemented in other exemplary embodiments as device features.

Representative, non-limiting examples of the present invention were described above in detail with reference to the attached drawings. This detailed description is merely intended to teach a person of skill in the art further details for practicing preferred aspects of the present teachings and is not intended to limit the scope of the invention. Furthermore, each of the additional features and teachings disclosed above may be utilized separately or in conjunction with other features and teachings to provide improved an improved method of testing a ceramic component.

Moreover, combinations of features and steps disclosed in the above detailed description may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

All features disclosed in the description and/or the claims are intended to be disclosed separately and independently from each other for the purpose of original written disclosure, as well as for the purpose of restricting the claimed subject matter, independent of the compositions of the features in the embodiments and/or the claims. In addition, all value ranges or indications of groups of entities are intended to disclose every possible intermediate value or intermediate entity for the purpose of original written disclosure, as well as for the purpose of restricting the claimed subject matter.

### REFERENCE NUMBER LIST

1 Method

2 Heating or cooling

3 Heating or cooling

4 Testing

5 Component

6 Component

What is claimed is:

1. A method for testing a ceramic component for a fracture toughness, comprising:
changing the temperature of the component to a first temperature; and
changing the temperature of the component to a second temperature; wherein a temperature difference between the first temperature and the second temperature is determined based on a minimum fracture toughness; and
testing the component for cracks.

2. The method according to claim 1, wherein a value for the minimum fracture toughness is taken from a table.

3. The method according to claim 1, wherein the measured fracture toughness is at most +/−10% of a minimum required fracture toughness.

4. The method according to claim 1, wherein the fracture toughness is determined based on:

| | h_{f} = 75000 Wm⁻²K⁻¹ | | | h_{f} = 100000 Wm⁻²K⁻¹ | | |
|---|---|---|---|---|---|---|
| | Bi | Kₛᵤᵣᵥ | K_{frac} | Bi | Kₛᵤᵣᵥ | K_{frac} |
| | — | MPa m^{1/2} | MPa m^{1/2} | — | MPa m^{1/2} | MPa m^{1/2} |
| Set 1, D = 12.7 mm, HK10 | 22.0 | 5.3 ± 0.3 | 5.5 ± 0.3 | 29.3 | 5.7 ± 0.3 | 5.9 ± 0.3 |
| Set 2, D = 5.55 mm, HK10 | 10.2 | 5.5 ± 0.3 | 5.9 ± 0.3 | 13.7 | 6.0 ± 0.3 | 6.4 ± 0.3 |
| Set 3, D = 5.55 mm, HK7 | 10.6 | 5.7 ± 0.1 | 6.0 ± 0.2 | 14.2 | 6.2 ± 0.1 | 6.5 ± 0.2 |

5. The method according to claim 1, wherein testing the component for cracks comprises applying a black dye to the component.

6. The method according to claim 1, further comprising determining a fracture toughness of a test component that corresponds to the ceramic component in shape, size, and material.

7. A ceramic component, manufactured using the method according to claim 1.

8. The ceramic components according to claim 7, wherein the ceramic component comprises more than 50% by weight of the material Si₃N₄, SiAlON, SiC, Al₂O₃, ZrO₂, or of their mixtures.

9. The ceramic components according to claim 7, wherein the ceramic component has a fracture toughness that is greater than or equal to 4 MPa√m.

10. The ceramic components according to claim 7, wherein the ceramic component has a roughness at least sectionally on its surface that is less than 15 μm.

11. The method according to claim 1, wherein changing the temperature of the component to the first temperature comprises heating the component and wherein changing the temperature of the component to the second temperature comprises cooling the component.

12. The method according to claim 1,
wherein the measured fracture toughness is at most +/−10% of a minimum required fracture toughness,
wherein the fracture toughness is determined based on:

| | h_{f} = 75000 Wm⁻²K⁻¹ | | | h_{f} = 100000 Wm⁻²K⁻¹ | | |
|---|---|---|---|---|---|---|
| | Bi | Kₛᵤᵣᵥ | K_{frac} | Bi | Kₛᵤᵣᵥ | K_{frac} |
| | — | MPa m^{1/2} | MPa m^{1/2} | — | MPa m^{1/2} | MPa m^{1/2} |
| Set 1, D = 12.7 mm, HK10 | 22.0 | 5.3 ± 0.3 | 5.5 ± 0.3 | 29.3 | 5.7 ± 0.3 | 5.9 ± 0.3 |
| Set 2, D = 5.55 mm, HK10 | 10.2 | 5.5 ± 0.3 | 5.9 ± 0.3 | 13.7 | 6.0 ± 0.3 | 6.4 ± 0.3 |
| Set 3, D = 5.55 mm, HK7 | 10.6 | 5.7 ± 0.1 | 6.0 ± 0.2 | 14.2 | 6.2 ± 0.1 | 6.5 ± 0.2 |

and, wherein testing the component for cracks comprises applying a black dye to the component.
